(19) United States
(12) Patent Application Publication
(10) Pub. No.: US 2012/0083650 A1
(43) Pub. Date: Apr. 5, 2012

(54) SYSTEMS AND METHODS FOR ADJUSTING GASTRIC BAND PRESSURE

(75) Inventor: Joseph S. Raven, Goleta, CA (US)

(73) Assignee: ALLERGAN, INC., Irvine, CA (US)

(21) Appl. No.: 12/894,718

(22) Filed: Sep. 30, 2010

(51) Int. Cl. A61F 2/00

(52) U.S. Cl. 600/37

(57) ABSTRACT

The present invention provides for an obesity treatment system for use in conjunction with a gastric band suitable for laparoscopic placement around a stomach of a patient to create a stoma. The obesity treatment system may use real-time objective measurement and clinical data to provide an optimal gastric band adjustment for the patient. The obesity treatment system may include a pressure sensing device coupled to the gastric band, and configured to detect a maximum tolerable pressure, and a pressure changing device coupled to the gastric band, and configured to adjust the gastric band for asserting an optimal pressure against the stomach of the patient, the optimal pressure based on an optimal pressure percentage and the maximum tolerable pressure.

### FIELD

The present invention generally relates to medical systems and apparatus and uses thereof for treating obesity and/or obesity-related diseases, and more specifically, relates to systems and methods for adjusting gastric band pressure.

### BACKGROUND

Adjustable gastric banding apparatus have provided an effective and substantially less invasive alternative to gastric bypass surgery and other conventional surgical weight loss procedures. Despite the positive outcomes of invasive weight loss procedures, such as gastric bypass surgery, it has been recognized that sustained weight loss can be achieved through a laparoscopically-placed gastric band, for example, the LAP-BAND® (Allergan, Inc., Irvine, Calif.) gastric band or the LAP-BAND AP® (Allergan, Inc., Irvine, Calif.) gastric band. Generally, gastric bands are placed about the cardia, or upper portion, of a patient's stomach forming a stoma that slows down the passage of food into the lower portion of the stomach. When the stoma is of an appropriate size, the tension created by the gastric band and the stomach wall tension created by the passage of food provides a feeling of satiety or fullness, which may discourage overeating. Unlike gastric bypass procedures, the gastric band apparatus are reversible and require no permanent modification to the gastrointestinal tract.

Over time, a stoma created by a gastric band may need adjustment in order to maintain an appropriate size, which should be neither too restrictive nor too passive. For example, an overly tight gastric band may cause the food to remain above the gastric band, which may likely to result in adverse events and pathologies like esophageal dilatation. Conventional gastric band adjustment systems and methods may adopt a subjective approach in adjusting a gastric band. For example, a physician or a care taker may arbitrarily increase or decrease the size of the stoma without first ascertaining the gastric band pressure. As such, conventional gastric band adjustment process may neglect the current physiological conditions of the patient and/or statistical data which may be applied to the patient's physiological conditions. Consequentially, the conventional adjustment process may or may not achieve a desirable weight loss result within a fixed period of time, and it may introduce undesirable side effects as well.

Attempts have been made in the past to use pressure as a guide pole for adjusting the gastric band. For example, Birk, U.S. Pub. No. 2007/01560131, discloses a system and a method for adjusting a gastric band by altering the pressure of the fluid contained therein. However, the system or the method disclosed in Birk may still be arbitrary because it does not take into account the current physiological conditions of an individual patient and it does not employ statistical data obtained from a clinical study to optimize the result of the adjustment process. Therefore, there is a need for a system and/or a method that may implement a gastric band pressure adjustment process which takes into account a patient's current physiological conditions as well as statistical data obtained from one or more groups of clinical subjects with successful weight loss results.

### SUMMARY

Generally described herein are systems and methods for adjusting gastric band pressure. The apparatus, systems and methods described herein may use real-time objective measurement and clinical data as adjustment parameters for adjusting gastric band pressure. Advantageously, the gastric band adjustment processes described herein may take into account each patient's current physiological conditions as well as statistical data obtained from a group of clinical subjects with successful weight loss results.

In one embodiment, the present invention may include an obesity treatment system for use in conjunction with a gastric band suitable for laparoscopic placement around a stomach of a patient to create a stoma. The obesity treatment system may use real-time objective measurement and clinical data to provide an optimal gastric band adjustment for the patient. The obesity treatment system may include a pressure sensing device coupled to the gastric band, and configured to detect a maximum tolerable pressure asserted by the gastric band against the stomach of the patient, and a pressure changing device coupled to the gastric band, and configured to adjust the gastric band for asserting an optimal pressure against the stomach of the patient, the optimal pressure based on an optimal pressure percentage and the maximum tolerable pressure.

In another embodiment, the present invention may include an obesity treatment system for use in conjunction with a gastric band suitable for laparoscopic placement around a stomach of a patient to create a stoma. The obesity treatment system may use real-time objective measurement and clinical data to provide optimal gastric band adjustment for the patient. The obesity treatment system may include an external controller configured to calculate an optimal pressure percentage, and telemetrically transmit an adjustment signal carrying an adjustment command based on the optimal pressure percentage, and an implantable controller coupled to the gastric band, configured to receive and process the adjustment signal, thereby retrieving the adjustment command, the implantable controller may have a pressure sensing device configured to detect a maximum tolerable pressure asserted by the gastric band against the stomach of the patient, and a pressure changing device configured to adjust the gastric band for asserting an optimal pressure against the stomach of the patient, the optimal pressure based on the adjustment command and the maximum tolerable pressure.

In another embodiment, the present invention may include an obesity treatment system, which may be used in conjunction with a gastric band suitable for laparoscopic placement around a stomach of a patient to create a stoma, for determining the patient's maximum tolerance to a pressure asserted by the gastric band against the patient's stomach. The obesity treatment system may include a pressure changing device configured to be coupled to the gastric band, the pressure changing device configured to repeatedly increase the pressure before the patient swallows a bolus at a step increment for every predetermined time period until a maximum tolerance event occurs, and a pressure sensing device configured to be coupled to the gastric band, the pressure sensing device configured to detect a maximum tolerable pressure when the maximum tolerance event occurs.

In another embodiment, the present invention may include an obesity treatment system for collecting, processing and utilizing clinical data from a plurality of patients, each of whom may be implanted with a gastric band suitable for laparoscopic placement around the patient's stomach to create a stoma. The obesity treatment system may include a memory configured to store a plurality of records, each record having an excess weight loss percentage and/or an adverse event rate relatable to at least one of the plurality of patients, and a processor, coupled to the memory, configured to create a target group of records from the plurality of records based on a weight loss criterion or a safety criterion, the weight loss criterion selecting the record having the excess weight loss percentage ranges from about 20% to about 50%, the safety criterion selecting the record having the adverse event rate at about 0.05% to about 0.01%, determine a normal distribution of the target group of records, and calculate an optimal pressure percentage based on the normal distribution.

In another embodiment, the present invention may include a method for treating obesity, which may be used in conjunction with a gastric band suitable for laparoscopic placement around a stomach of a patient to create a stoma. The method may use real-time objective measurement and clinical data to provide optimal gastric band adjustment for the patient. The method may include the steps of detecting a maximum tolerable pressure asserted by the gastric band against the stomach of the patient, receiving an optimal pressure percentage based on a plurality of clinical data from a plurality of subjects using a plurality of subject gastric bands, and adjusting the gastric band for asserting an optimal pressure against the stomach of the patient, the optimal pressure based on the optimal pressure percentage and the maximum tolerable pressure.

In still another embodiment, the present invention may include a gastric band adjustment device, which may be used in conjunction with a gastric band suitable for laparoscopic placement around a stomach of a patient to create a stoma, the gastric band having a pressure sensing device for sensing a pressure asserted by the gastric band against the stomach of the patient and a pressure changing device for changing the pressure asserted by the gastric band. The gastric band adjustment device may include a memory configured to store an optimal pressure percentage value, and a processor, coupled to the memory, configured to receive the pressure detected by the pressure sensing device of the gastric band, derive a maximum tolerable pressure based on the pressure detected by the pressure sensing device and a maximum tolerance event, the maximum tolerable pressure representative of the patient's maximum tolerance to the pressure asserted by the gastric band, calculate an optimal pressure based on the optimal pressure percentage and the maximum tolerable pressure, generate a pressure adjustment command based on the optimal pressure, and remotely adjust the gastric band pressure by transmitting the pressure adjustment command to the pressure changing device of the gastric band via the external transceiver.

### BRIEF DESCRIPTION OF THE DRAWINGS

The features, objects, and advantages of the invention will become more apparent from the detailed description set forth below when taken in conjunction with the drawings, wherein:

FIG. 1 shows a block diagram of a gastric band pressure adjustment system according to an embodiment of the present invention;

FIG. 2 shows a flow diagram of a method for adjusting a pressure of an implantable gastric band according to an embodiment of the present invention;

FIG. 3 shows a flow diagram of a method for adjusting the pressure of an implantable gastric band by detecting a maximum tolerable pressure according to another embodiment of the present invention;

FIG. 4 shows a flow diagram of a method for determining an optimal pressure percentage according to an embodiment of the present invention;

FIG. 5 shows a flow diagram of another method for determining an optimal pressure percentage according to another embodiment of the present invention;

FIGS. 6A and 6B show a flow diagram of another method for adjusting the pressure of an implantable gastric band according to yet another embodiment of the present invention;

FIG. 7 shows a schematic view of an external gastric band pressure adjustment system according to an embodiment of the present invention;

FIG. 8A shows a schematic view of an automatic gastric band pressure adjustment system according to an embodiment of the present invention;

FIG. 8B shows a schematic view of another automatic gastric band pressure adjustment system according to an alternative embodiment of the present invention;

FIG. 9 shows a real time pressure chart of the inner band pressure of the gastric band during an initial adjustment according to an embodiment of the present invention;

FIG. 10A shows a schematic view of a system for determining various weight loss optimal pressure percentage values according to an embodiment of the present invention;

FIG. 10B shows various data fields of an exemplary clinical record according to an embodiment of the present invention;

FIG. 11 shows a schematic view of a system for determining various safety optimal pressure percentage values according to an embodiment of the present invention; and

FIGS. 12A-12B show a perspective view and a block diagram of a remote gastric band adjustment device according to an embodiment of the present invention.

### DETAILED DESCRIPTION

In accordance with exemplary embodiments, the present invention comprises various systems and method for adjusting a pressure of an implantable gastric band by utilizing real-time objective measurements and computer processed clinical data. Persons skilled in the art will readily appreciate that various aspects of the present invention may be realized by any number of methods and devices configured to perform the intended functions. Stated differently, other methods and devices may be incorporated herein to perform the intended functions. It should also be noted that the drawing FIGS. referred to herein are not all drawn to scale, but may be exaggerated to illustrate various aspects of the invention, and in that regard, the drawing FIGS. should not be construed as limiting. Finally, although the present invention may be described in connection with various medical principles and beliefs, the present invention should not be bound by theory.

By way of example, the present invention will be described primarily with references to hydraulically adjustable gastric bands. Nevertheless, persons skilled in the art will readily appreciate that the present invention advantageously may be applied to one of the numerous varieties of fluid filled surgical implants presently comprising, or which may in the future comprise, access ports. Similarly, while the present invention will be described primarily with reference to fluid filled surgical implants, persons skilled in the art will readily appreciate that the present invention advantageously may be applied to other devices, and whether fluid or gel filled.

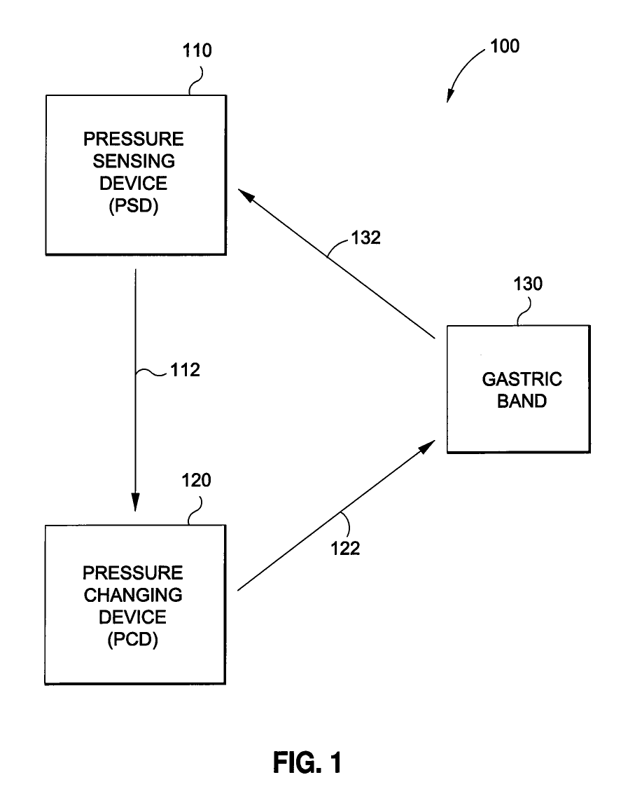

The discussion now begins with FIG. 1, which shows a block diagram of a gastric band pressure adjustment system 100 according to an embodiment of the present invention. Generally, the gastric band pressure adjustment system 100 may include a pressure sensing device (PSD) 110, a pressure changing device (PCD) 120, and a pressure adjustable gastric band 130. Persons skilled in the art may readily appreciate that the gastric band 130 may be implanted inside a patient's body, and it may be suitable for laparoscopic placement around the patient's stomach for creating a stoma thereof.

In one aspect of the current embodiment, the PSD 110 and the PCD 120 may be in fluid communication with the gastric band 130. As such, the PSD 110 may sense an inner band pressure 132 of a fluid contained within the gastric band 130. Because the gastric band 130 may wrap around the patient's stomach to create the stoma thereof, the inner band pressure 132 may reflect the pressure asserted by the gastric band 130 against the patient's stomach as well as the pressure asserted by the patient's stomach against the gastric band 130. Hence, the PSD 110 may sense an active band pressure, which may be asserted by the gastric band 130, and a reactive pressure, which may be induced by the stomach of the patient.

In another aspect of the current embodiment, the PSD 110 may be utilized to detect a maximum tolerable pressure (MTP) 112 of the patient, which may be received and/or used by the PCD 120 for adjusting the band pressure of the gastric band. More specifically, the PCD 120 may adjust the inner band pressure 132 to an optimal pressure 122, which may be a function of the MTP 112 and a clinically derived optimal pressure percentage (OPP) value.

The pressure adjustment system 100 may provide several advantages over conventional pressure adjustment systems. For example, by measuring or sensing the MTP 112 from a particular patient at real-time, the gastric band pressure adjustment system 100 may take into account the patient's current physiological conditions in an objective fashion. Thus, the gastric band pressure adjustment system 100 may normalize the inner band to accommodate to the patient's current physiological conditions.

For another example, by using the clinically derived optimal pressure percentage value, the gastric band pressure adjustment system 100 may incorporate statistical data from clinical studies with the subjects that may have attained successful weight loss and/or safety results. Advantageously, the gastric band pressure adjustment system 100 may increase a likelihood of achieving successful weight loss results for a particular patient. As such, the gastric band pressure adjustment system 100 may also reduce the need of frequent band readjustment.

The discussion now turns to various methods for adjusting the implantable gastric band according to various embodiments of the present invention. In one aspect, these methods may be implemented by the gastric band pressure adjustment system 100. In another aspect, these methods may be implemented by various gastric band pressure adjustment systems, which will be discussed in later sections.

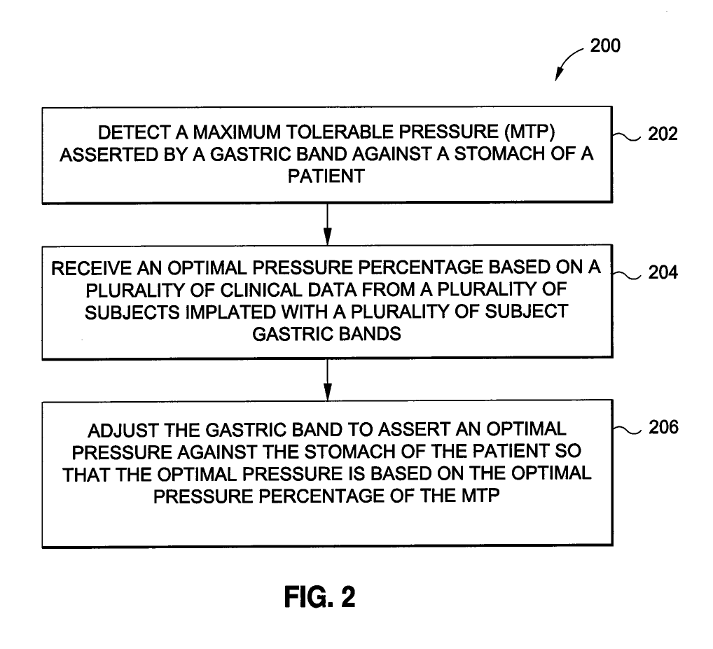

FIG. 2 shows a flow diagram of a method 200 for adjusting the implantable gastric band according to an embodiment of the present invention. In step 202, a maximum tolerable pressure (MTP) may be detected. Generally, the MTP may be representative of the patient's maximum tolerance to the pressure asserted by the gastric band.

In one embodiment, the MTP may be a pressure asserted by the gastric band against the stomach of the patient, and at which the patient may experience a general discomfort. To manifest such general discomfort, the patient may exhibit one or more symptoms that may be the result of wearing an overly tight gastric band. More specifically, the one or more symptoms may include but are not limited to a lower thoracic tightness, nausea, stomach obstruction, regurgitation, reflux, and/or pressured back throat.

In another embodiment, the MTP may be a pressure asserted by the gastric band which causes the stoma of the patient's stomach to be substantially closed. As a result, an esophagus or the stomach of the patient may react to the substantial closure of the stoma by inducing a series of pressure spikes. In most instances, the series of pressure spikes may be redirected to the upper surface of the stoma, and against the gastric band.

In any event, the MTP may be a predefined pressure not exceeding a gastric band pressure threshold, standard, or limit set by the United States Food and Drug Administration (U.S.F.D.A.). In one embodiment, such predefined pressure may be based on a patient's most recent gastric band adjustment history. In another embodiment, such predefined pressure may be based on a trend of the patient's gastric band adjustment history. In another embodiment, such predefined pressure may be an iso-static pressure in relation to atmospheric pressure. In yet another embodiment, such predefined pressure may be based on a real-time objective measurement of the patient's physiological conditions.

Referring to step 204, an optimal pressure percentage may be received. Generally, the optimal pressure percentage may be based on a plurality of clinical data collected from a plurality of subjects, who may be implanted with a plurality of subject gastric bands for a predefined period of time. Among other benefits, the optimal pressure percentage may provide a successful and reproducible methodology for optimal gastric band adjustment.

In one embodiment, the optimal pressure percentage may be a median pressure percentage or a mean pressure percentage of the plurality of subjects who may experience successful weight loss results. As defined herein, the pressure percentage of a particular subject may be a ratio of the subject's average band pressure over the subject's maximum tolerable pressure.

In another embodiment, the optimal pressure percentage may be a median pressure percentage or a mean pressure percentage of the plurality of subjects who may have the least occurrence of adverse events. As discussed herein, the adverse event may include but is not limited to hiatal hernia, bleeding, symmetrical pouch dilatation, death, and/or serious bodily injury.

In step 206, the gastric band may be adjusted to assert an optimal pressure against the patient's stomach. In one embodiment, the optimal pressure may be a function of the patient's MTP and the optimal pressure percentage received from the clinical data. In another embodiment, the optimal pressure may be a product of the patient's MTP multiplied by the optimal pressure percentage. In yet another embodiment, the optimal pressure may be a weighted product of the patient's MTP multiplied by various optimal pressure percentages.

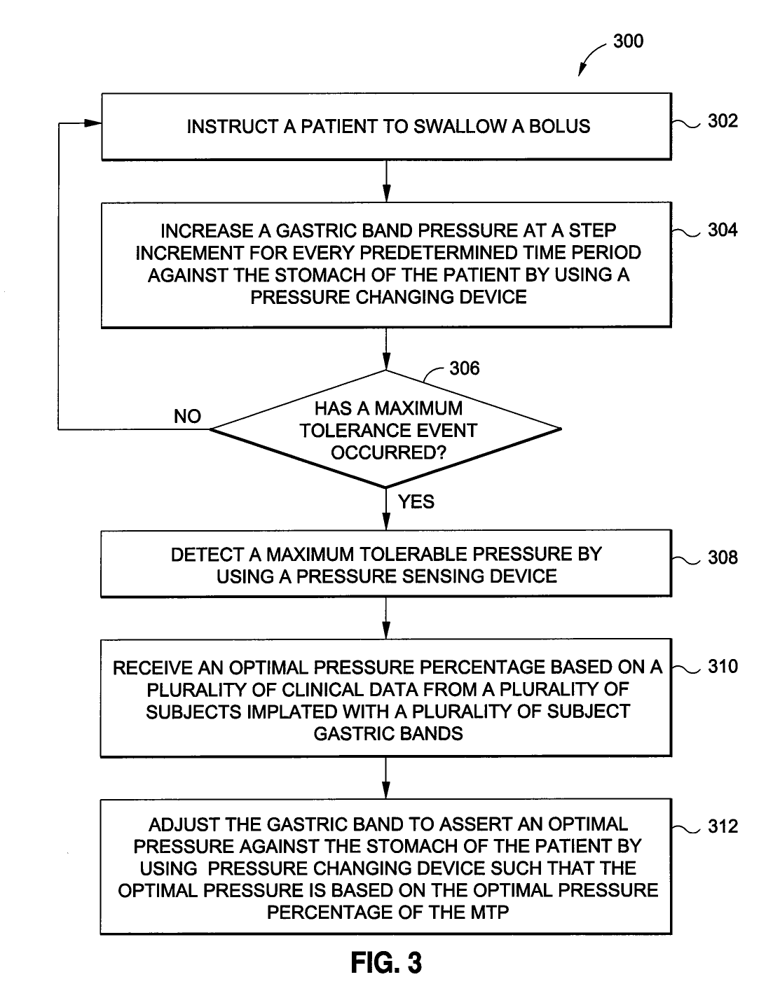

The discussion now turns to FIG. 3, which shows a method 300 for adjusting the pressure of a gastric band by detecting a maximum tolerable pressure (MTP). According to an embodiment of the present invention, the MTP may be a real-time objective measurement of a particular patient's physiological conditions. In step 302, the patient implanted with the gastric band may be instructed to swallow a bolus. Persons skilled in the art may readily appreciate that the term “bolus” may refer to various ingestible objects that are safe for human consumption. For example, the bolus may include a small amount of water, liquid food and/or solid food.

In step 304, the inner band pressure of the implanted gastric band may be increased at a step increment for every predetermined time period before the patient swallows the bolus. In an embodiment of the present invention, the step increment may range, for example, from about 0.1 psi to about 3 psi, and the predetermined time period may range, for example, from about 20 seconds to about 4 minutes. In another embodiment of the present invention, the step increment may range, for example, from about 0.2 psi to about 2 psi, and the predetermined time period may range, for example, from about 30 seconds to about 3 minutes. In yet another embodiment of the present invention, the step increment may range, for example, from about 0.3 psi to about 1 psi, and the predetermined time period may range, for example, from about 1 minute to about 2 minutes.

In step 306, a maximum tolerance event may occur if the gastric band pressure begins to upset the patient's stomach or esophagus. If the maximum tolerance event occurs in step 306, the gastric band adjustment method 300 may proceed to step 308. If the maximum tolerance event is yet to occur in step 306, step 302 and step 304 may be repeated accordingly.

As defined herein, the maximum tolerance event may be an event that may indicate that the patient's maximum tolerance to the pressure asserted by the gastric band has reached. In one embodiment of the present invention, for example, the maximum tolerance event may occur when the patient begins to experience lower thoracic tightness, nausea, stomach obstruction, regurgitation, reflux, and/or back throat pressure. In another embodiment of the present invention, for example, the maximum tolerance event occurs when the stomach or esophagus of the patient induces a series of reactive pressure spikes against the gastric band. In yet another embodiment of the present invention, for example, the maximum tolerance event occurs when the inner band pressure or volume of the gastric band reaches the U.S.F.D.A. predefined threshold.

In step 308, the maximum tolerable pressure (MTP) may be detected by using a pressure sensing device. In one embodiment, the pressure sensing device may be a pressure transducer. In another embodiment, the pressure sensing device may be a volume gauge. In yet another embodiment, the pressure sensing device may be an optical sensor. Regardless of the type of pressure sensing device used, the MTP may be detected by measuring the inner band pressure of the gastric band during or shortly before the occurrence of the maximum tolerance event.

In step 310, an optimal pressure percentage may be received. In step 312, the gastric band pressure may be adjusted to an optimal pressure based on the detected MTP and the received optimal pressure percentage. According to an embodiment of the present invention, steps 310 and 312 may be similar to steps 204 and 206 of the method 200 as discussed in FIG. 2. As such, the descriptions and advantages related to steps 204 and 206 may equally be applicable to steps 310 and 312, respectively.

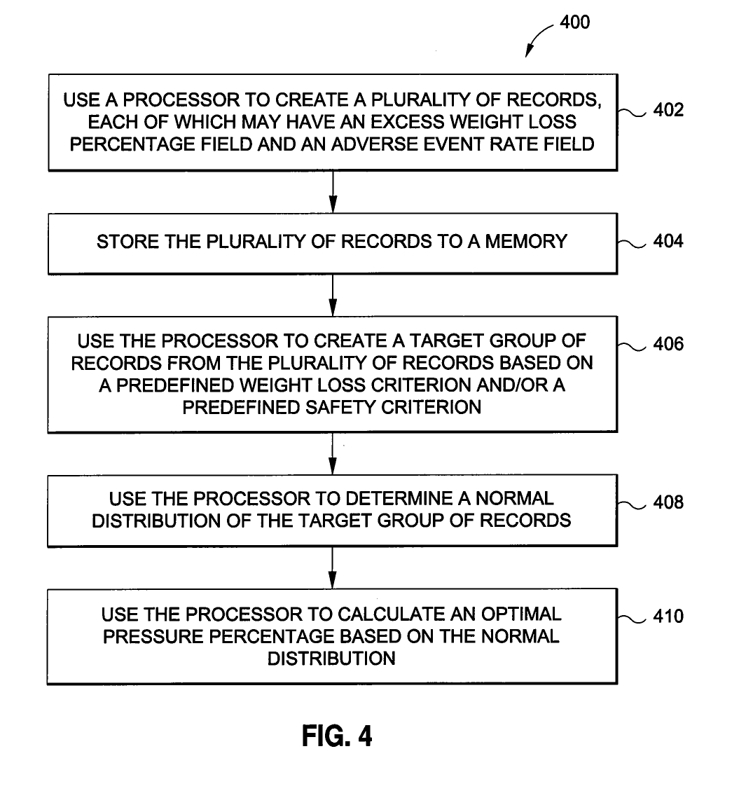

The discussion now turns to FIG. 4, which shows a flow diagram of a method 400 for determining an optimal pressure percentage according to an embodiment of the present invention. In step 402, a processor may be used to create a plurality of records. Generally, each of the record may contain information pertinent to a particular subject who participates in a clinical study for gastric band usages. Particularly, the clinical study may focus on the correlation between the pressure percentages of subject gastric bands, which may be worn by a group of subjects, and the weight loss and safety results achieved by the group of subjects within about one year after they are implanted with the subject gastric bands. More specifically, each of the records may contain an excess weight loss percentage field and an adverse event rate field. In one embodiment, the excess weight loss percentage field may contain a ratio of a total weight loss over an initial excess weight. As defined herein, the initial excess weight is a difference between the subject's initial weight and a normal person's weight, and the total weight loss is a difference between the initial weight and a current weight of the subject. For example, assuming that the subject weighs 300 pound initially and a normal person may weigh 180 pounds, and that the subject currently weighs 240 pounds, the subject may have an initial excess weight of 120 pounds and a total weight loss of 60 pounds. Accordingly, the subject may have an excess weight loss percentage of 50%. In another embodiment, the adverse event rate field may contain a number of occurrence(s) of adverse event as well as the type of adverse event that may have occurred.

In step 404, the plurality of records may be stored to a memory, which may be coupled to and accessible by the processor. As defined herein, the processor can be any computing device capable of receiving data, processing the received data, and outputting the processed data. For example, the processor can be coupled to a display and the memory. The processor may be implemented using hardware, software, firmware, middleware, microcode, or any combination thereof. The processor may be an Advanced RISC Machine (ARM), a computer, a controller, a digital signal processor (DSP), a microprocessor, circuitry, a processor chip, or any other device capable of processing data, and combination thereof. The memory may include or store various routines and data. The term “memory” includes, but is not limited to, random access memory (RAM), flash memory, read-only memory (ROM), EPROM, EEPROM, registers, hard disk, removable disk, CD-ROM, DVD, Blu-ray disk, wireless channels, and various other media capable of storing, containing or carrying instruction(s) and/or data. The display may be a CRT, LCD, LED, and/or plasma display screen or a touch screen.

In step 406, the processor may be used to create a target group of records from the plurality of records based on at least one predefined weight loss criterion and/or at least one predefined safety criterion. Specifically, the processor may search the plurality of records against each record's excess weight loss percentage field or adverse event rate field. If the data contained in a record's weight loss field or adverse event rate field satisfy the weight loss criterion and/or the safety criterion, the processor may select that record and copy it to the target group of records. Although the weight loss criterion and the safety criterion are used for selecting the plurality of records in this embodiment, other criteria may be used for creating the target group of records as well according to various embodiments of the present invention. For example, each of the plurality of records may contain a body fat ratio field which may record a particular subject's body fat ratio, such that in step 406, a body fat criterion may be used in selecting the target group of records.

In step 408, the processor may be used to determine a normal distribution of the target group of records. Particularly, the processor may determine the correlation between the pressure percentages and the excess weight loss percentage. That is, the processor may use the data stored in the target group of records to determine what pressure percentage range may achieve the most desirable weight loss results without jeopardizing the health of the patients. For example, the processor may execute a statistic application to determine an average excess weight loss percentage, a normal distribution of the excess weight loss percentage, and/or a standard deviation of the pressure percentages against the excess weight loss percentages among the target group of records. Alternatively, the processor may execute statistic application to determine an average adverse event rate percentage, a normal distribution of the adverse event rate, and a standard deviation of the pressure percentages against the adverse event rate among the target group of records.

In step 410, the processor may be used to calculate an optimal pressure percentage based on the normal distribution of the excess weight loss percentage or the normal distribution of the adverse event rate. According to an embodiment of the present invention, the optimal pressure percentage may be related to and categorized under certain physiological parameters pertinent to the target group of subjects. For example, the physiological parameters may include but are not limited to a range of maximum tolerable pressure (MTP) values, a range of blood pressures, a range of blood glucose levels, age group, sex group, pregnancy status, history of cancer, and/or history of cardio arrest. Advantageously, the optimal pressure percentage derived herein may be used for determining the optimal pressure in the gastric band pressure adjustment methods 200 and 300.

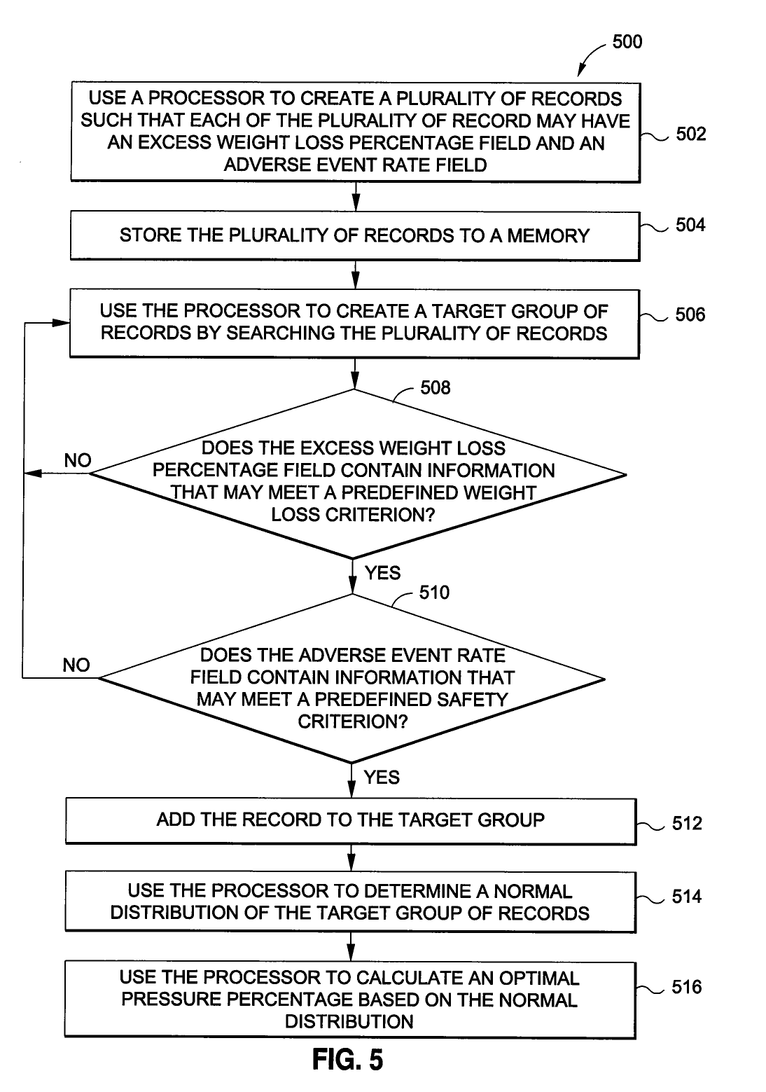

The discussion now turns to FIG. 5, which shows a flow diagram of another method 500 for determining an optimal pressure percentage according to another embodiment of the present invention. Initially, the method 500 may include similar steps as the method 400. For example, step 502 may be similar to step 402, and step 504 may be similar to step 404. As such, steps 502 and 504 may be described and understood in similar fashion as steps 402 and 404.

In step 506, the processor may be used to create a target group of records by searching the plurality of records. In step 508, the processor may check each of the plurality of records to ascertain whether the excess weight loss percentage field contains information that may meet a predefined weight loss criterion. If the excess weight loss percentage field does not contain any information that may meet the predefined weight loss criterion, the processor may return to step 506 to search for the next record from the plurality of records. Alternatively, if the excess weight loss percentage field contains information that meets the predefined weight loss criterion, the processor may continue the screening process and proceed to step 510.

The predefined weight loss criterion may be set by the processor and/or by a human operator. Generally, the predefined weight loss criterion may be used as an indicator to show that a particular subject has attained successful weight loss results since the inception of the subject gastric band implantation. In one embodiment of the present invention, the predefined weight loss criterion may be a range of excess weight loss percentage values, which may range, for example, from about 5% to about 60%. In another embodiment of the present invention, the predefined weight loss criterion may be a range of excess weight loss percentage values, which may range, for example, from about 20% to about 50%. In yet another embodiment of the present invention, the predefined weight loss criterion may be a range of excess weight loss percentage values, which may range, for example, from about 30% to about 40%.

In step 510, the processor may check the passing record from step 508 to ascertain whether the adverse event rate field thereof contains information that may meet a predefined safety criterion. If the adverse event rate field does not contain any information that may meet the predefined safety criterion, the processor may return to step 506 to search for the next record from the plurality of records. Alternatively, if the adverse event rate field contains information that meets the predefined safety criterion, the process may proceed to step 512.

The predefined safety criterion may be set by the processor and/or by a human operator. Generally, the predefined safety criterion may be used as an indicator to show that a particular subject has attained successful safety results since the inception of the subject gastric band implantation. More specifically, the adverse events may include but is not limit to hiatal hernia, bleeding, symmetrical pouch dilatation, death, and/or serious bodily injury.

The information contained in the adverse event rate field may be specific to a particular subject, or it may be specific to a sub-group of subjects who may share similar physiological parameters, such as race, sex, pregnancy status, maximum tolerable pressures, smoking habit, drinking habit, cancer history, and/or cardiovascular history. In one embodiment, for example, the information contained in the adverse event rate field may record the number of adverse events occurred to the particular subject within a fixed period of time. In another embodiment, for example, the information contained in the adverse event rate field may record the frequency of occurrence of the adverse event among a sub-group of subjects, each of which may have a pressure percentage value that is within a close range of pressure percentages values (e.g., a 5% range, a 10% range, a 15% range, and/or a 20% range). In another embodiment, for example, the information contained in the adverse event rate field may record the frequency of occurrence of the adverse event among another sub-group of subjects, each of which may have a MTP within a close range of the maximum tolerable pressure values (e.g., a 0.5 psi range, a 1.0 psi range, a 2 psi range, and/or a 4 psi range).

In that regard, a subject with a low adverse event rate may have achieved a desirable or successful safety result. Similarly, a sub-group of subjects sharing a low adverse event rate may indicate that these subjects have jointly achieved a desirable or successful safety result, meaning that the adjustment setting of these subjects' gastric bands are unlikely to bring forth the adverse event.

In one embodiment of the present invention, the predefined safety criterion may be a range of adverse event rate values, which may range, for example, from about 0.5% to about 0.00001%. In another embodiment of the present invention, the predefined safety criterion may be a range of adverse event rate values, which may range, for example, from about 0.1% to about 0.0001%. In yet another embodiment of the present invention, the predefined safety criterion may be a range of adverse event rate value, which may range, for example, from about 0.05% to about 0.01%.

After the selection steps 506-510 are performed, the processor may proceed to step 512, which may add the passing record to the target group of records. In step 514, the processor may be used to determine a normal distribution of the target group of records. Because step 514 is similar to step 408 of the method 400, the description and advantages of step 408 may be applicable to step 514. In step 516, the processor may be used to calculate an optimal pressure percentage based on the normal distribution determined in step 514. Because step 516 is similar to step 410 of the method 400, the description and advantages of step 410 may be applicable to step 516.

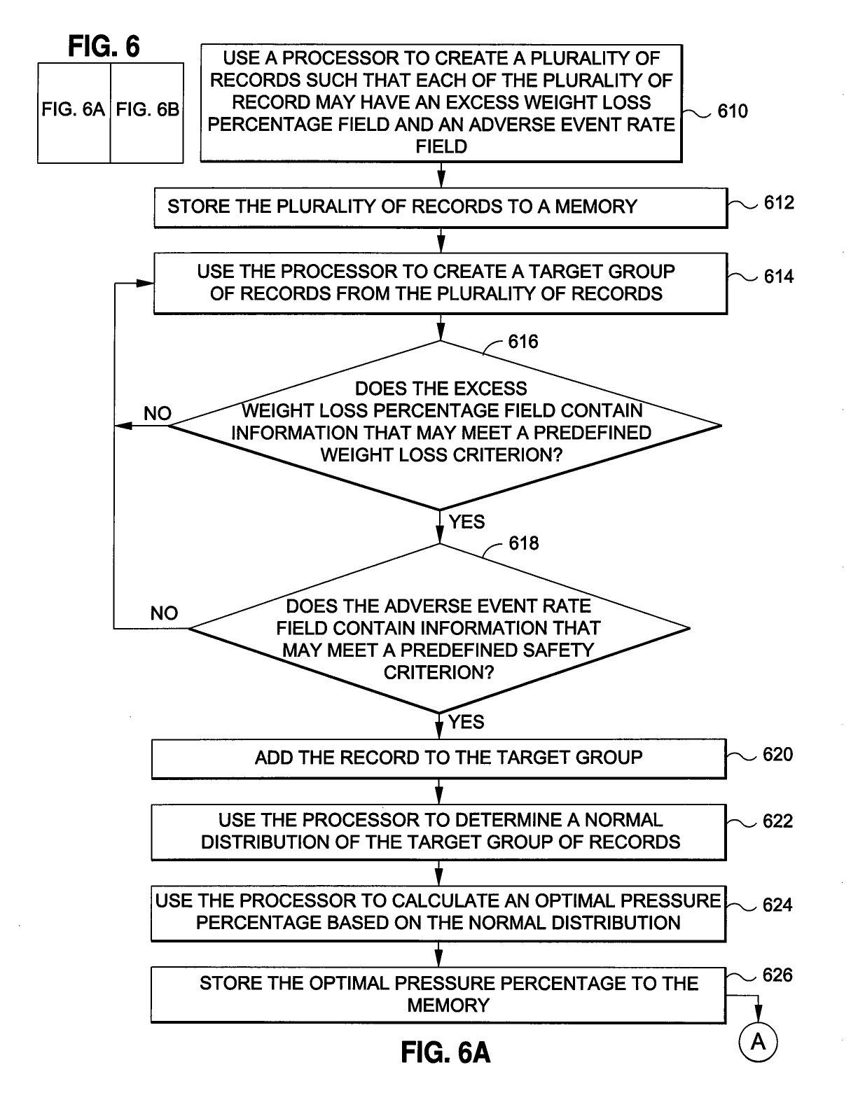

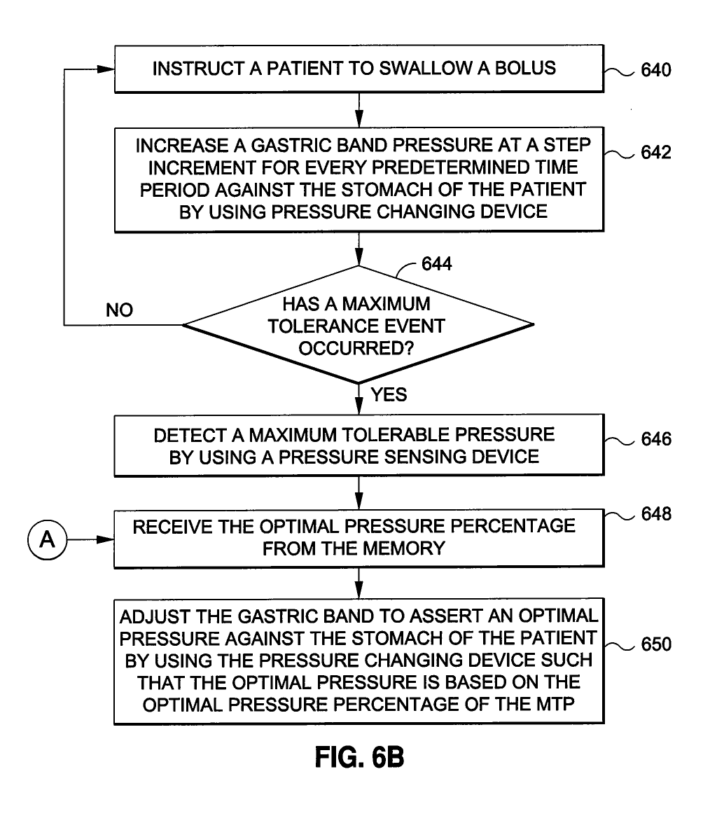

The discussion now turns to FIGS. 6A and 6B, which show a flow diagram of another method 600 for adjusting the pressure of an implantable gastric band according to an embodiment of the present invention. Generally, the method 600 may include two concurrent threads. As shown in FIG. 6A, the first thread may include steps 610 to 626, which may be similar to the steps of method 500. As shown in FIG. 6B, the second thread may include steps 640 to 650, which may be similar to the steps of the method 300. Therefore, the description and advantages of steps 302 to 312 may be applicable to steps 640 to 650 and the description and advantages of steps 502 to 516 may be applicable to steps 610 to 624.

More specifically, the first thread may include method steps for determining an optimal pressure percentage. In step 610, a processor may be used to create a plurality of records, each of which may have an excess weight loss percentage field and an adverse event rate field.

In step 612, the plurality of records may be stored in a memory. As persons skilled in the art may readily appreciate, the excess weight loss percentage field and the adverse event rate field may be constantly, periodically, iteratively, or randomly updated during the course of the clinical study, so that changes to the data contained in these two fields may be properly received by the memory and/or processed by the processor.

In step 614, the processor may be used to create a target group of records from the plurality of records. In step 616, the processor may be used to ascertain whether the information contained in the excess weight loss percentage field of a particular record may meet a predefined weight loss criterion. In step 618, the processor may be used to ascertain whether the information contained in the adverse event rate field of a particular record may meet a predefined safety criterion.

If the particular record passes both steps 616 and 618, it may be added to the target group of records in step 620. Otherwise, the processor will not select that particular record and return to step 614 to screen the next record. The predefined weight loss criterion and the predefined safety criterion may share similar properties or characteristics with the weight loss criterion and the predefined safety criterion discussed in FIG. 5. Moreover, the predefined weight loss criterion and the predefined safety criterion may encompass additional steps not mentioned in FIG. 5.

In step 622, the processor may be used to determine a normal distribution of the target group of records. In step 624, the processor may be used to calculate an optimal pressure percentage based on the normal distribution. In step 626, the optimal pressure percentage may be stored in the memory. It is worth noting that the first thread of method steps may be initiated, repeated, or updated as frequently as it may be desired. For example, the first thread of method steps may be initiated one or more months after the subjects have been implanted with the subject gastric bands. For another example, the first thread of method steps may be repeated for every other month, every three months, every six months, or every twelve months. For yet another example, the first thread of method steps may be updated when one or more new subjects join the clinical study, as soon as an adverse event is reported, or upon request by a third party user.

Next the second thread may include method steps for optimizing a patient's gastric band pressure by using the optimal pressure percentage derived from the method steps of the first thread and by using a maximum tolerable pressure of the patient. In step 640, the patient may be instructed to swallow a bolus. In step 642, a pressure changing device may be used to increase the gastric band pressure at a step increment for every predetermined time period while the patient is swallowing the bolus and until a maximum tolerance event may occur during step 644. Next, in step 646, a pressure sensing device may be used to detect the maximum tolerable pressure. In step 648, the optimal pressure percentage may be received from the memory. If the above steps are implemented by a human care taker (or physician), the care taker may request the processor to perform an updated calculation as discussed in steps 610 to 626. Alternatively, if the above steps are implemented by an automated medical device, the automated medical device may obtain the optimal pressure percentage from a shared network hard drive or any other shared storage medium. In step 648, the pressure changing device may be used to adjust the gastric band pressure to an optimal pressure, which may be based on the optimal pressure percentage of the maximum tolerable pressure.

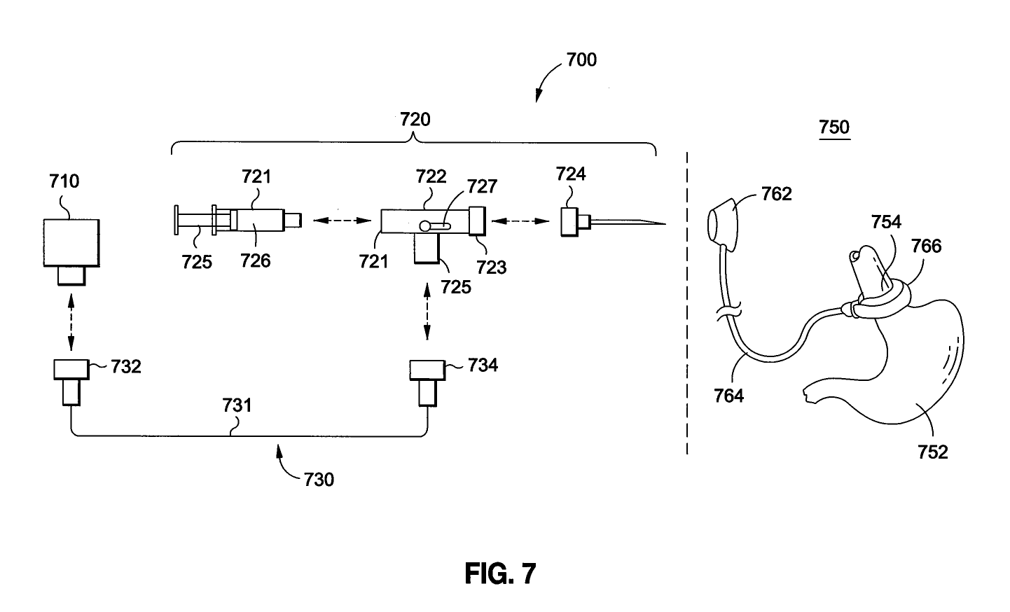

The discussion now turns to various systems and devices for implementing or executing the methods 200, 300, 400, 500, and/or 600 as discussed in FIGS. 2-6. FIG. 7 shows a schematic view of an external gastric band pressure adjustment system 700 according to an embodiment of the present invention. Generally, the external gastric band pressure adjustment system 700 may be used when a patient 750 is implanted with a fluid filled gastric band 766, which may be connected to an access port 762 via a tube 764. Particularly, the access port 762, the tube 764, and the fluid filled gastric band 766 may be in fluid communication such that they may share the same pressure internally. The access port 762 may be used to maintain the pressure within an inflatable portion of the fluid filled gastric band 766 by limitedly allowing access to the fluid retained thereof.

More specifically, the inflatable portion of the fluid filled gastric band 766 may be placed around a stomach 752 of a patient 750 to form a stoma 754, such that the inflatable portion of the fluid filled gastric band 766 may be used to control a size of the stoma. Usually, the stoma 754 may have a relatively small size when the pressure within the inflatable portion is relatively high, whereas the stoma 754 may have a relatively large size when the pressure within the inflatable portion is relatively small.

In one embodiment, the external gastric band pressure adjustment system 700 may include a pressure sensing device (PSD) 710, a pressure changing device (PCH) 720, and a connecting device 730. The PSD 710 may be a pressure transducer which may sense a pressure of a certain amount of fluid having a finite volume. The connecting device 730 may have a tube 731 coupled between a first receiving port 732 and a second receiving port 734, such that the first receiving port 732 may be coupled to the PSD 710.

The PCH 720 may have an adjustable volume chamber 721, a multi-port device 722 and an insertion device 724. Particularly, the adjustable volume chamber 721 may be used to adjust the pressure of the inflatable portion of the fluid filled gastric band 766. For example, the adjustable volume chamber 721 may be a syringe with a piston 725 and a chamber 726 with a volume adjustable by the piston. The insertion device 724 may be used for accessing the access port 762, which may be implanted inside the patient 750. As such, the insertion device 724 may be a needle capable of inserting through the patient's skin, subcutaneous fat, and eventually a septum of the access port 762. The multi-port device 722 may include first, second, and third ports 723, 725, and 721, and a handle 727 for controlling the connectivity among the first, second, and third ports 723, 725, and 721.

As shown in FIG. 7, the multi-port device 722 may have its first port 723 coupled to the insertion device 724, second port 725 coupled to the second receiving port 734 of the connecting device 730, and third port 721 coupled to the adjustable volume chamber 721. After these ports are properly connected, the external gastric band pressure adjustment system 700 may be used to adjust the pressure of the fluid filled gastric band 766.

In detecting a maximum tolerable pressure of the patient 750, the PSD 710 may be used in conjunction with the PCD 720. Initially, for example, the PCD 720 may be used to empty the fluid contained within the fluid filled gastric band 766 via the access port 762, such that the inflatable portion of the fluid filled gastric band 766 may assert a minimum pressure against the stomach 752 of the patient 750. Accordingly, the stoma 754 may have a first size. Next, for example, the PCD 720 may be used to steadily increase the pressure of the fluid filled gastric band 766 by injecting fluid thereto via the access port 762 until a maximum tolerable event may occur.

Meanwhile, the PSD 710 may be used to monitor the pressure of the fluid injected into the fluid filled gastric band 766, and it may be used to detect the maximum tolerable pressure when the maximum tolerance event occurs. After detecting the maximum tolerable pressure, the PCH 720 may, for example, be used to adjust the pressure of the fluid stored inside of the fluid filled gastric band 766 to the optimal pressure, which may be based on the clinically derived optimal pressure percentage and the currently detected maximum tolerable pressure.

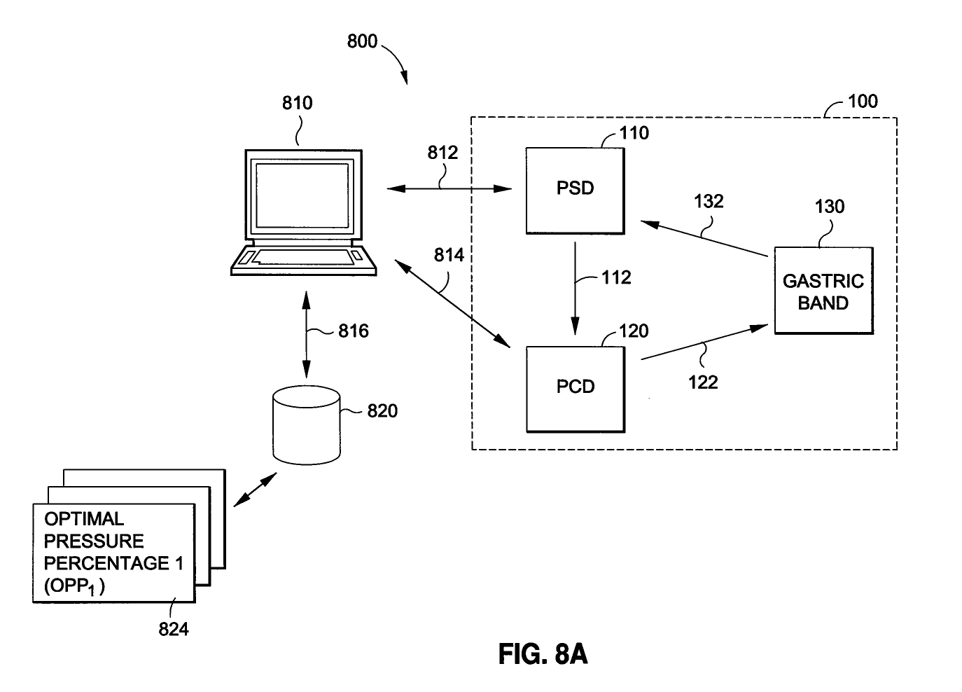

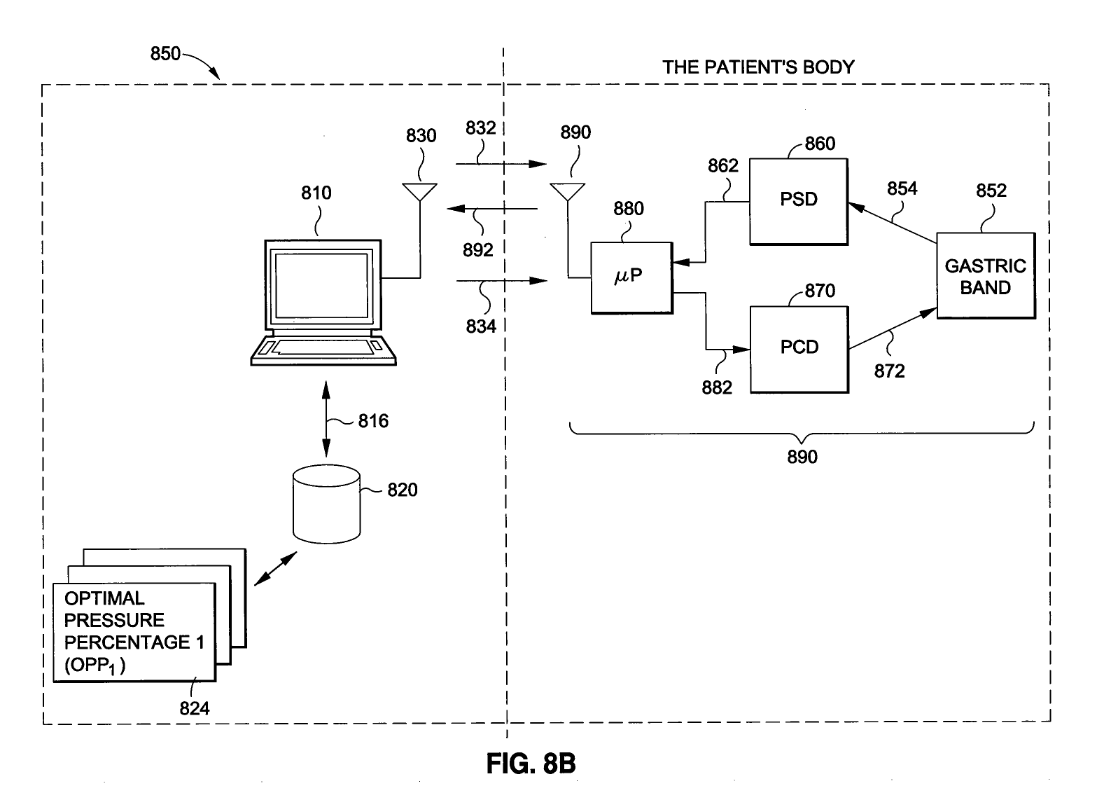

The discussion now turns to FIG. 8A, which shows a schematic view of an automatic gastric band pressure adjustment system 800 according to an embodiment of the present invention. Generally, the automatic gastric band pressure adjustment system 800 may incorporate the basic gastric band pressure adjustment system 100 as shown in FIG. 1. Moreover, the automatic gastric band pressure adjustment system 800 may include a processor 810 and a memory 820, which may be accessible and rewritable by the processor 810. Particularly, the processor 810 may be coupled to the pressure sensing device (PSD) 110 via a first connection 812 and the pressure changing device (PCD) 120 via a second connection 814.

As defined herein, the processor 810 can be any computing device capable of receiving data, processing the received data, and outputting the processed data. For example, the processor 810 can be coupled to a display and a memory. The processor 810 may be implemented using hardware, software, firmware, middleware, microcode, or any combination thereof. The processor 810 may be an Advanced RISC Machine (ARM), a computer, a controller, a digital signal processor (DSP), a microprocessor, circuitry, a processor chip, or any other device capable of processing data, and combination thereof. The memory 820 may include or store various routines and data. The term “memory” includes, but is not limited to, random access memory (RAM), flash memory, read-only memory (ROM), EPROM, EEPROM, registers, hard disk, removable disk, CD-ROM, DVD, Blu-ray disk, wireless channels, and various other media capable of storing, containing or carrying instruction(s) and/or data. The display may be a CRT, LCD, LED, and/or plasma display screen or a touch screen. The first and second connections 812 and 814 may be established via an electrical wire, an electromagnetic coupling, an infra red communication system, a Bluetooth communication system, a fiber optic wire, and/or other communication media suitable for telemetric communication.

In one embodiment of the present invention, the memory 820 may store a plurality of optimal pressure percentage (OPP) values 824, each of which may be derived from a clinical study related to gastric band adjustment optimization. Particularly, each of clinical studies may be directed to a group of subjects with similar physiological conditions. For example, a study directed to a group of subjects with a high range of maximum tolerable pressure (MTP) values may derive one OPP value. For another example, another study directed to a group of subjects with a low range of MTP values may derive another OPP value. For yet another example, a study directed to a group of subjects suffering from high blood pressure conditions may derive yet another OPP value.

To begin the gastric band pressure adjustment process, the processor 810 may first determine the physiological conditions of a current patient, who may be implanted with a gastric band 130. Depending on the gastric band system being used, the gastric band 130 may be integrated with the PSD 110 and PCD 120, or it may be a stand alone device. Accordingly, the PSD 110 and the PCD 120 may be located outside the patient's body, or alternatively, they may be implanted inside the patient's body.

In one embodiment, the processor 810 may instruct the PSD 110 to cooperate with the PCD 120 to determine the patient's MTP by executing a series of steps consistent with the methods 200, 300, 400, 500, and 600 as discussed in FIGS. 2-6. For example, the processor 810 may instruct the PCD 120 to increase the band pressure of the gastric band 130 with incremental steps and it may, at the same time, instruct the PSD 110 to sense and transmit the inner band pressure 132 of the gastric band 130 back to the processor 810 for processing. Advantageously, the processor 810 may simultaneously adjust the gastric band pressure and monitor for the occurrence of the maximum tolerable event.

Once the processor 810 detects the occurrence of a maximum tolerance event, it may determine the MTP of the patient. Consequentially, the processor 810 may access the memory 820 via a connection 816 to retrieve an OPP value that meets the patient's physiological profile, which may include the patient's real-time MTP measurement result.

Next, the processor 810 may calculate an optimal pressure for the patient based on the patient's MTP and the retrieved OPP. For example, the optimal pressure may be a product of the MTP multiplied by the OPP. That is, the optimal pressure may be the optimal pressure percentage of the maximum tolerable pressure. For another example, the optimal pressure may be a weighted product of the MTP and the OPP.

After calculating the optimal pressure for the patient, the processor 810 may send another instruction to the PCD 120 to adjust the pressure of the gastric band to the optimal pressure 122.

In an alternative embodiment, the processor 810 may simply initiate the MTP measurement procedure by sending an instruction to both the PSD 110 and PCD 120, each of which may be automated or programmed to perform one or more of the method steps as discussed in FIGS. 2-6. As such, the PSD 110 and the PCD 120 may determine the MTP value of the patient, and the PSD 110 may communicate the MTP value back to the processor 810. Upon receiving the MTP value, the processor 810 may proceed to calculate the optimal pressure for the patient by using the MTP value and the OPP value, which may be retrieved from the memory 820. After that, the processor 810 may send yet another instruction to the PCD 120 to adjust the pressure of the gastric band 130 to the optimal pressure 122.

The discussion now turns to FIG. 8B, which shows a schematic view of another automatic gastric band pressure adjustment system 850 according to an alternative embodiment of the present invention. Generally, the system 850 may be similar to the system 800 in several aspects. For example, the system 850 may include the processor 810 and the memory 820, which may store several OPP values 824. For another example, the PSD 860 may be in fluid communication with the gastric band 852, and it may be used to sense the inner band pressure 854 of the gastric band 852. For yet another example, the PCD 870 may be coupled to the gastric band 852, and it may be used to adjust the band pressure of the gastric band to the optimal pressure 872.

Despite these similarities, the system 850 may be different from the system 800 in at least one aspect. For example, the system 850 may include an integrated gastric band system 890, which may incorporate the PSD 860, the PCD 870, and a microprocessor 880 to the gastric band 852. Specifically, the processor 810 may have a transceiver 830, which may telemetrically communicate with an internal transceiver 890 of the microprocessor 880. More specifically, the microprocessor 880 may be preprogrammed to execute one or more of the method steps discussed in FIGS. 2-6 upon receiving an initiation request 832 from the processor 810.

In order to perform the MTP measurement process, the microprocessor 880 may communicate and interact with the PSD 860 via a first connection 862 and with the PCD 870 via a second connection 882. According, the microprocessor 880 may simultaneously adjust the gastric band pressure and monitor for the occurrence of the maximum tolerable event.

After the MTP measurement process is completed, the microprocessor 880 may request for an appropriate OPP value from the processor 810. Specifically, the microprocessor 880 may submit the MTP as an operand in the request command 892. Upon receiving the request command 892 from the microprocessor 880, the processor 810 may retrieve the matching OPP by searching the memory 820. When the processor 810 locates the matching OPP value, it may transmit back to the microprocessor 880 a confirmation command 834, which may be embedded with the OPP value. Consequentially, the microprocessor 880 may calculate the optimal pressure 872 based on one or more method steps as discussed in FIGS. 2-6. Next, the microprocessor 880 may instruct the PCD 820 to adjust the gastric band with the optimal pressure 872.

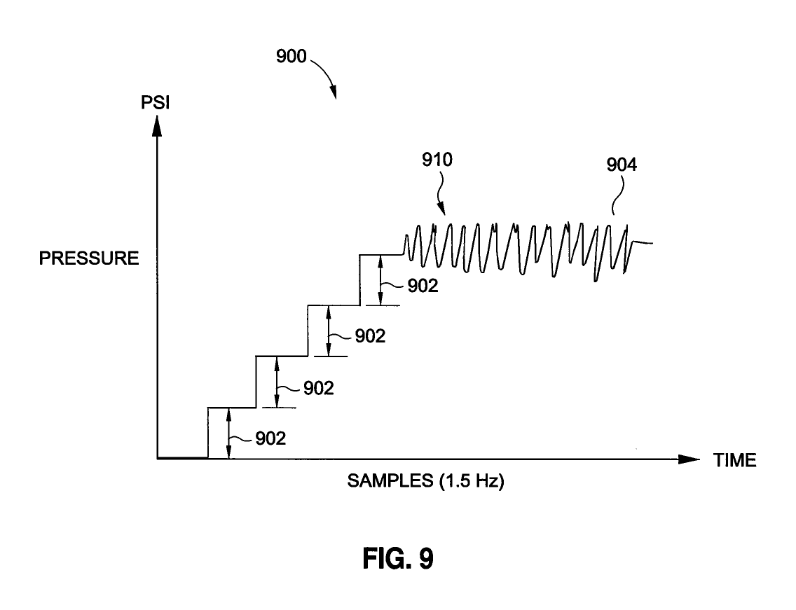

The discussion now turns to FIG. 9, which shows a real time pressure chart 900 of the inner band pressure of the gastric band during an initial adjustment. Generally, the processor 810 may be connector to a display, which may display in real-time the measured inner band pressure of the gastric band in the format of the pressure chart 900. As previously discussed, the initial adjustment process may involve measuring a patient's maximum tolerable pressure (MTP) to ascertain the patient's physiological conditions, which may be indicated by the patient's maximum tolerance to the pressure asserted by the gastric band against the patient' stomach.

Among other aforementioned methods and techniques, the MTP may be observed and measured by steadily increasing the inner band pressure, while the patient is swallowing a bolus, which may be a small quantity of ingestible liquid or solid food. At the outset, the gastric band may be partially or completely evacuated so that the inner band pressure may be substantially reduced. Then, the patient may begin to swallow the bolus, and the inner band pressure may be increased simultaneously. In one embodiment, the gastric band pressure may be increased continuously and linearly over a period of time while the patient is swallowing the bolus. In another embodiment, the gastric band pressure may be increased in step increments 902 as shown in FIG. 9.

When the pressure asserted by the gastric band is bearable to the patient, the stomach and/or the esophagus of the patient may function normally and may unlikely react to the increased gastric band pressure. As the pressure asserted by the gastric band increases, the patient may feel less comfortable and eventually, the stomach and/or esophagus of the patient may begin to react to the increasing gastric band pressure. Particularly, when the pressure asserted by the gastric band becomes unbearable to the patient's stomach, which may or may not be felt by the patient, the stomach and/or esophagus may induces a series of rapid pressure spikes against the gastric band.

As a result, the inner band pressure may be influenced by the series of rapid pressure spikes, so that the inner band pressure may fluctuate with the rapid pressure spikes. Such fluctuation may be sensed by the pressure sensing device, processed by the processor, and eventually output by the display as a series of measured pressure spikes 904. As discussed previously, the series of measured pressure spikes 904 may manifest the occurrence of a maximum tolerance event 910, which may indicate that the inner band pressure of the gastric band has reached the particular patient's maximum tolerable pressure.

Because each patient may have a different set of physiological conditions, and because the rate of weight loss and/or the frequency of occurrence of adverse event may be closely related to the set of physiological conditions, it is important and advantageous that the gastric band adjustment process may take into account the patient's current physiological conditions before performing the actual adjustment. By introducing the MTP to the gastric band adjustment process, the gastric band adjustment methods and systems discussed herein may help accelerate the rate of weight loss and reduce the rate of occurrence of an adverse event.

The discussions now turns to various systems that may be used to derive one or more optimal pressure percentage (OPP) values by implementing one or more of the methods 200, 300, 400, 500, and 600 as discussed in FIGS. 2-6.

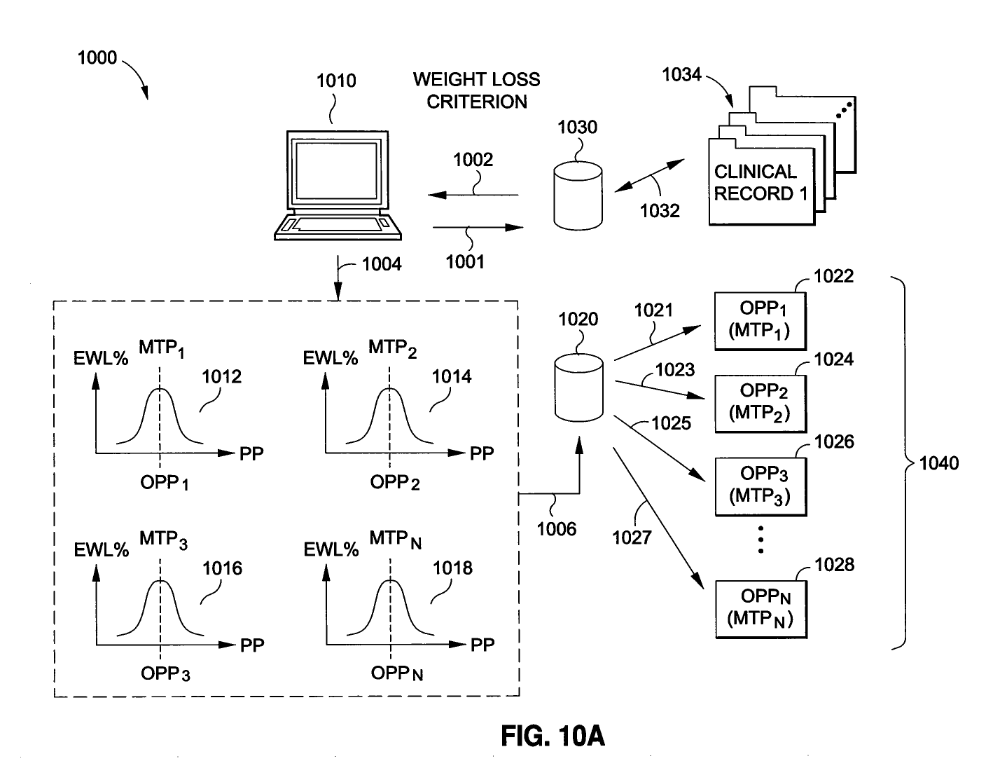

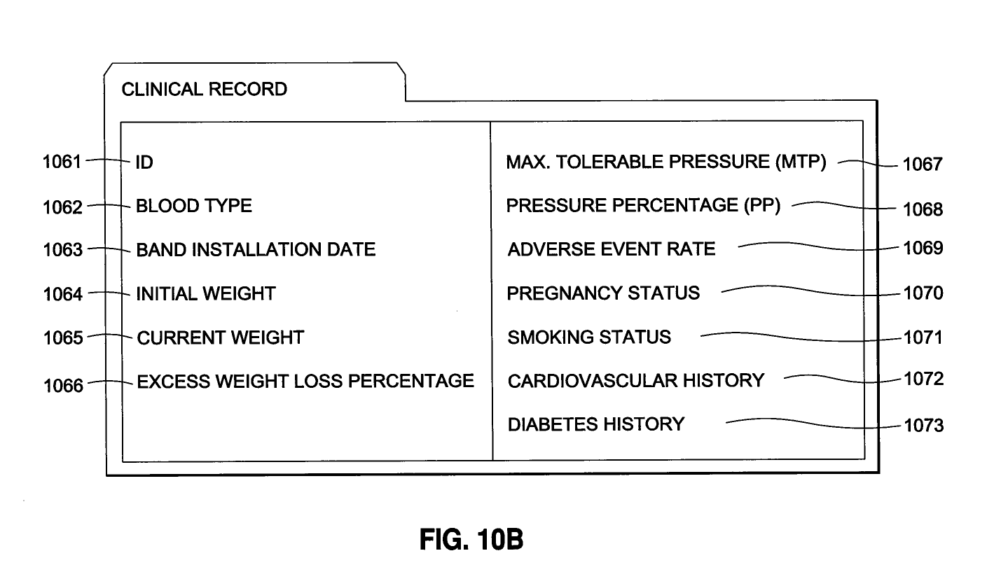

FIG. 10A shows a schematic view of a system 1000 for determining various weight loss optimal pressure percentage values according to an embodiment of the present invention. Generally, the system 1000 may include a processor 1010, which may be similar to the processor 810 as shown and discussed in FIGS. 8A-8B, a first memory 1030, and a second memory 1020. Both the first and second memories 1030 and 1020 may be implemented by hardware similar to those discussed with respect to the memory 820 and as shown in FIGS. 8A-8B. Particularly, the first memory 1030 may be configured to store a plurality of clinical records 1342, and the second memory 1020 may be configured to store a plurality of optimal pressure percentage (OPP) values 1040. Furthermore, both the first and second memories 1030 and 1020 may be accessed, searched, written and rewritten by the processor 1010 via wired and/or wireless connections.

In one embodiment, each of the clinical records 1034 may be associated with a clinical study group subject who may be implanted with a subject gastric band. The plurality of clinical records 1034 may each adopt a record format similar to that of an exemplary clinical record 1060 as shown in FIG. 10B. Generally, the exemplary clinical record 1060 may include several data fields for recording information related to the subject's identity, weight loss progress, gastric band adjustment setting, and other physiological parameters.

For example, the exemplary clinical record 1060 may include an ID field 1061 for recording the identity of the subject, a blood type field 1062 for recording the blood type of the subject, a band installation date field 1063 for recording the date when the subject gastric band is implanted in the subject, an initial weight field 1064 for recording the initial weight of the subject before implanted with the subject gastric band, a current weight field 1065 for recording a current weight of the subject, an excess weight loss percentage field 1066 for recording the current excess weight loss percentage of the subject, a maximum tolerable pressure (MTP) filed 1067 for recording the average or current MTP of the subject, a pressure percentage (PP) field 1068 for recording a ratio of the current subject gastric band pressure over the MTP of the subject, an adverse event rate field 1069 for recording the number of occurrences of an adverse event(s) of the subject or of a sub-group of subjects having similar physiological parameters as the subject, a smoking status field 1070 for recording the number of cigarette(s) the subject may consume on a weekly basis, a cardiovascular history field 1072 for recording the subject's cardiovascular history, and a diabetes history field 1073 for recording the subject's diabetes history.

Referring again to FIG. 10A, the processor 1010 may initially create the plurality of clinical records 1034 and have them stored in the first memory 1030. The processor 1010 may periodically, iteratively or randomly access and update the first memory 1030 by sending a first instruction 1001 to the first memory 1030. The first memory 1030 may execute a search-and-update function 1032 to locate and rewrite the clinical records 1034.

Each time when the processor 1010 is used to calculate one or more OPP values 1040, the processor 1010 may first accept one or more screening criteria, which may include but is not limited to the predefined weight loss criterion and/or the safety criterion as discussed in the methods 400, 500, and 600. Then, the processor 1010 may perform a screening task, which may compare the appropriate data fields (e.g., the excess weight loss percentage field 1066 and the adverse event rate field 1069) with the one or more screening criteria. As shown in FIG. 10A, the weight loss criterion may be used to screen the clinical records 1034 according to an embodiment of the present invention.

Once a match is found, the matched clinical record 1002 may be sent back to the processor 1010. The processor 1010 may then copy and add the matched clinical record 1002 to a target group of records, which may be stored in a temporary storage medium accessible by the processor 1010. The screening process may be iterated until all the clinical records 1034 are screened or until a sub-group of clinical records 1034 are screened.

Next, the processor 1010 may execute a series of statistical calculation 1004 to determine one or more normal distributions of the target group records. Generally, each of the normal distributions may be represented by a plot of pressure percentage (PP) values against excess weight loss percentage (WL %) values.

For example, the processor 1010 may determine a first normal distribution 1012, which may be related to a subset of target group records, the subjects in which may each have a low maximum tolerable pressure MTP₁. The processor 1010 may then use the first normal distribution 1012 to compute or calculate a first OPP (OPP₁), which may take into account the MTP₁ as a physiological parameter.

For another example, the processor 1010 may determine a second normal distribution 1014, which may be related to a subset of target group records, the subjects in which may each have a medium maximum tolerable pressure MTP₂. The processor 1010 may then use the second normal distribution 1014 to compute or calculate a second OPP (OPP₂), which may take into account the MTP2 as a physiological parameter.

For another example, the processor 1010 may determine a third normal distribution 1016, which may be related to a subset of target group records, the subjects in which may each have a high maximum tolerable pressure MTP₃. The processor 1010 may then use the third normal distribution 1016 to compute or calculate a third OPP (OPP₃), which may take into account of the MTP3 as a physiological parameter.

For yet another example, the processor 1010 may determine an Nth normal distribution 1018, which may be related to a subset of target group records, the subjects in which may each have a specific range of maximum tolerable pressure MTP_{N}. The processor 1010 may then use the Nth normal distribution 1018 to compute or calculate an Nth OPP (OPP_{N}), which may take into account of the MTPN as a physiological parameter.

After the OPP values are calculated, the processor 1010 may send an update command 1006 to the second memory 1020. The second memory 1020 may then perform a rewrite function 1021 for updating the value of the OPP₁, a rewrite function 1023 for updating the value of the OPP₂, a rewrite function 1025 for updating the value of the OPP₃, and a rewrite function 1027 for updating the value of the OPP_{N}. In an embodiment of the present invention, the first and second memories 1030 and 1020 may be implemented in one piece of hardware. Alternatively, the first and second memories 1030 and 1020 may be implemented in two or more pieces of stand alone hardware in another embodiment of the present invention.

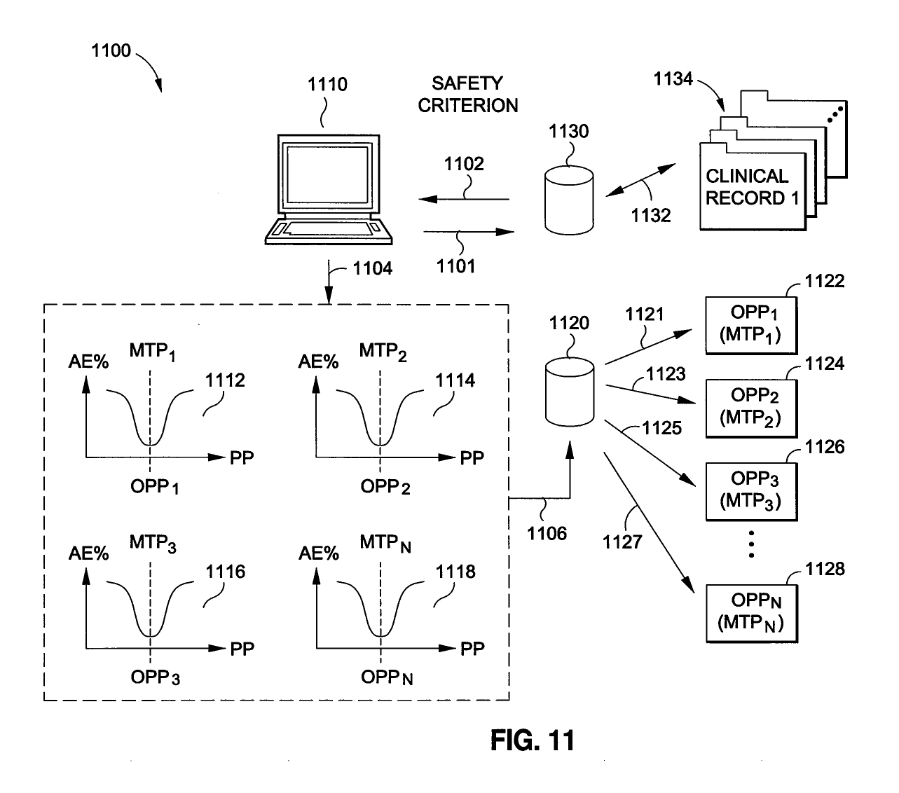

The discussion now turns to FIG. 11, which shows a schematic view of a system 1100 for determining various safety optimal pressure percentage values according to an embodiment of the present invention. Generally, the system 1100 may be similar to the system 1000 except that the system 1100 may use a safety criterion for screening the clinical records 1134.

The processor 1110 may initially create the plurality of clinical records 1134 and have them stored in the first memory 1130. The processor 1110 may periodically, iteratively or randomly access and update the first memory 1130 by sending a first instruction 1101 to the first memory 1130. The first memory 1130 may execute a search-and-update function 1132 to locate and rewrite the clinical records 1134.

Next, the processor 1110 may perform a screening task, which may compare the adverse event rate field 1069 with the safety criterion. The safety criterion may be similar to the one or more safety criteria as discussed in the methods 400, 500, and 600. Once a match is found, the matched clinical record 1102 may be sent back to the processor 1110. The processor 1110 may then copy and add the matched clinical record 1102 to a target group of records, which may be stored in a temporary storage medium accessible by the processor 1110. The screening process may be iterated until all the clinical records 1134 are screened or until a sub-group of clinical records 1134 are screened.

After the screening process is completed, the processor 1110 may execute a series of statistical calculation 1104 to determine one or more normal distributions of the target group records. Generally, each of the normal distributions may be represented by a plot of pressure percentage (PP) values against adverse event rate (AE %) values.

Next, the processor 1110 may determine first, second, and third normal distributions 1112, 1114, and 1116, each of which may be related to a subset of target group records. Particularly, each subset of target group records may include subjects with either one of the high, medium or low maximum tolerable pressure MTP₁, MTP₂, and MTP₃. Accordingly, the processor 1110 may then use the first, second, and third normal distributions 1112, 1114, and 1116 to compute or calculate the respective first, second, and third optimal pressure percentages OPP₁, OPP₂, and OPP₂ values.

Alternatively, the processor 1110 may determine an Nth normal distribution 1118, which may be related to a subset of target group records, the subjects in which may each have a specific range of maximum tolerable pressure MTP_{N}. The processor 1110 may then use the Nth normal distribution 1118 to compute or calculate an Nth OPP (OPP_{N}), which may take into account the MTP_{N} as a physiological parameter.

After determining the one or more OPP values, the processor 1110 may send an update command 1106 to the second memory 1120. The second memory 1120 may then perform a rewrite function 1121 for updating the value of the OPP₁, a rewrite function 1123 for updating the value of the OPP₂, a rewrite function 1125 for updating the value of the OPP₃, and a rewrite function 1127 for updating the value of the OPP_{N}.

Although FIGS. 10A and 11 show that the systems 1000 and 1100 are two isolated systems, the systems 1000 and 1100 may be integrated into one single system in an alternative embodiment of the present invention. Accordingly, the processor of the integrated system may screen the clinical records with a plurality of criteria, which may include both the weight loss criterion and the safety criterion. As a result, the processor of the integrated system may generate a target group of records that may satisfy both the weight loss criterion and the safety criterion, such that the optimal pressure percentage derived therefrom may be used for achieving optimized weight loss result and safety result.

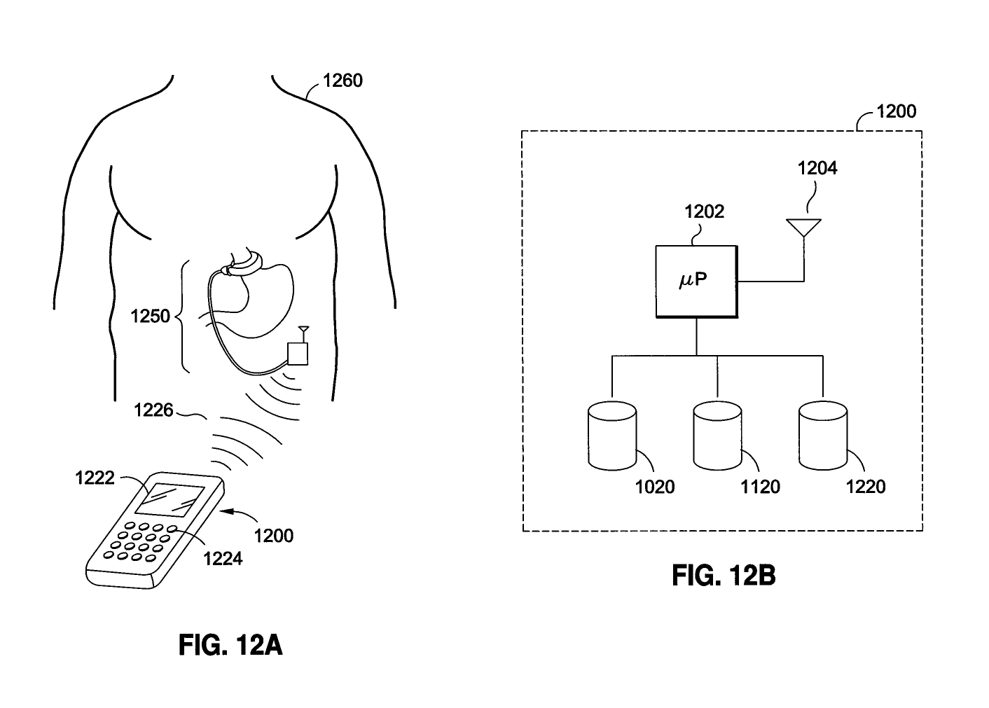

The discussion now turns to FIGS. 12A and 12B, which show a perspective view and a block diagram of a remote gastric band adjustment device (RGBAD) 1200 according to an embodiment of the present invention. Generally, the RGBAD 1200 may be used to adjust an integrated gastric band system 1250, which may include components and features that are similar to the integrated gastric band system 890 as shown in FIG. 8B. The RGBAD 1200 may have a display unit 1222, which may display information pertinent to the gastric band adjustment process, a set of buttons 1224, which may be used to receive user input, a microprocessor 1202, and a transceiver 1204.

More specifically, the microprocessor 1202 may initiate a gastric band adjustment process upon request from a user. The microprocessor 1202 may instruct the integrated gastric band system 1250 to begin the adjustment process via a telemetric connection 1225 established between the transceiver 1204 and the integrated gastric band system 1250. After a maximum tolerable pressure (MTP) is detected or sensed, the integrated gastric band system 1250 may transmit the MTP value back to the RGBAD 1200 for retrieving an optimal pressure percentage (OPP) value, which may be related to the currently measured MTP value.

In return, the RGBAD 1200 may search for one or more OPP values that may be accustomed to the physiological profile of the patient 1260. For example, the microprocessor 1202 may access the second memory 1020, which may be used to store a series of weight loss OPP values, the second memory 1120, which may be used to store a series of safety OPP values, and/or another second memory 1220, which may be used to store a series of other predefined OPP values. In one embodiment, the second memories 1020, 1120, and 1220 may be incorporated to the RGBAD 1200 as shown in FIG. 12B. As such, the second memories 1020, 1120, and 1220 may be periodically or randomly updated to obtain the most recent OPP values. Alternatively, the second memories 1020, 1120, and 1220 may be accessible from a remote network and/or a removable storage medium.

After obtaining the OPP value, the processor 1202 may transmit the OPP value back to the integrated gastric band system 1250, which may calculate an optimal pressure for the patient 1260 and adjust the pressure of the gastric band accordingly.

The foregoing disclosure is illustrative of the present invention and is not to be construed as limiting the invention. Although one or more embodiments of the invention have been described, persons skilled in the art will readily appreciate that numerous modifications could be made without departing from the spirit and scope of the present invention. It should be understood that all such modifications are intended to be included within the scope of the invention.

The terms “a,” “an,” “the,” and similar referents used in the context of describing the present invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the present invention otherwise claimed. No language in the specification should be construed as indicating any non-claimed element essential to the practice of the present invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, certain references have been made to patents and printed publications throughout this specification. Each of the above-cited references and printed publications are individually incorporated herein by reference in their entirety.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the present invention disclosed herein are illustrative of the principles of the present invention. Other modifications that may be employed are within the scope of the present invention. Thus, by way of example, but not of limitation, alternative configurations of the present invention may be utilized in accordance with the teachings herein. Accordingly, the present invention is not limited to that precisely as shown and described.

What is claimed is:

1. An obesity treatment system for use in conjunction with a gastric band suitable for laparoscopic placement around a stomach of a patient to create a stoma, the obesity treatment system using real-time objective measurement and clinical data to provide an optimal gastric band adjustment for the patient, the obesity treatment system comprising:
a pressure sensing device coupled to the gastric band, and configured to detect a maximum tolerable pressure asserted by the gastric band against the stomach of the patient; and
a pressure changing device coupled to the gastric band, and configured to adjust the gastric band for asserting an optimal pressure against the stomach of the patient, the optimal pressure based on an optimal pressure percentage and the maximum tolerable pressure.

2. The system of claim 1, wherein the maximum tolerable pressure is an iso-static pressure asserted by the gastric band against the stomach of the patient.

3. The system of claim 1, wherein the maximum tolerable pressure is representative of the patient's tolerance to a pressure asserted by the gastric band.

4. The system of claim 3, wherein the maximum tolerable pressure is the pressure causing the stoma of the stomach of the patient to be substantially closed.

5. The system of claim 3, wherein the maximum tolerable pressure is the pressure causing the patient to experience a discomfort selected from a group consisting of lower thoracic tightness, nausea, stomach obstruction, regurgitation, reflux, pressured back throat and combinations thereof.

6. The system of claim 3, wherein the maximum tolerable pressure is the pressure causing the stomach or an esophagus of the patient to induce a series of reactive pressure spikes against the gastric band while the patient is swallowing a bolus, and wherein the pressure sensing device is configured to sense the series of reactive pressure spikes, thereby detecting the maximum tolerable pressure.

7. The system of claim 1, wherein the optimal pressure percentage is based on a normal distribution of a plurality of clinical data from a plurality of subjects, each implanted with a subject gastric band, having weight loss results range from about 20% to about 50% excess weight loss within about one year after implanted with the subject gastric bands.

8. The system of claim 7, wherein the weight loss results range from about 30% to about 40% excessive weight loss.

9. The system of claim 1, wherein the optimal pressure percentage is based on a normal distribution of a plurality of clinical data from a plurality of subjects, each implanted with a subject gastric band, having a total occurrence rate of hiatal hernia ranges from about 0.5% to about 0.00001%.

10. The system of claim 1, wherein the gastric band has:
an inflatable portion placed around the stomach of the patient, the inflatable portion configured to control a size of the stoma; and
an access port configured to maintain the pressure within the inflatable portion of the gastric band by retaining a fluid disposed therein.

11. The system of claim 10, wherein the pressure changing device comprises:
a needle having a tapered end and a base end, the tapered end of the needle configured to coupled to the inflatable portion of the gastric band via the access port,
a multi-port device having first, second, and third ports, the first port coupled to the base end of the needle, the second port coupled to the pressure sensing device, and
an adjustable volume chamber coupled to the third port of the multi-port device, and configured to add or remove the fluid from the inflatable portion of the gastric band, thereby adjusting the pressure therein.

12. An obesity treatment system for use in conjunction with a gastric band suitable for laparoscopic placement around a stomach of a patient to create a stoma, the obesity treatment system using real-time objective measurement and clinical data to provide optimal gastric band adjustment for the patient, the system comprising:
an external controller configured to calculate an optimal pressure percentage, and telemetrically transmit an adjustment signal carrying an adjustment command based on the optimal pressure percentage; and
an implantable controller coupled to the gastric band, configured to receive and process the adjustment signal, thereby retrieving the adjustment command, the implantable controller having:
a pressure sensing device configured to detect a maximum tolerable pressure asserted by the gastric band against the stomach of the patient, and
a pressure changing device configured to adjust the gastric band for asserting an optimal pressure against the stomach of the patient, the optimal pressure based on the adjustment command and the maximum tolerable pressure.

13. The system of claim 12, wherein the maximum tolerable pressure is a pressure, asserted by the gastric band against the stomach of the patient, causing the stoma to be substantially closed.

14. The system of claim 12, wherein the maximum tolerable pressure is a pressure, asserted by the gastric band against the stomach of the patient, causing the patient to experience a discomfort selected from a group consisting of lower thoracic tightness, nausea, stomach obstruction, regurgitation, reflux, pressured back throat and combinations thereof.

15. The system of claim 12, wherein the maximum tolerable pressure is a pressure, asserted by the gastric band against the stomach of the patient, causing the stomach or esophagus of the patient to induce a series of reactive pressure spikes against the gastric band while the patient is swallowing a bolus, and wherein the pressure sensing device is configured to sense the series of reactive pressure spikes, thereby detecting the maximum tolerable pressure.

16. The system of claim 12, wherein the optimal pressure percentage is based on a normal distribution of a plurality of clinical data from a plurality of subjects, each implanted with a subject gastric band, and each having:
a subject gastric band pressure based on the subject's maximum tolerable pressure;
a weight loss result ranges from about 30% to about 40% excessive weight loss within about one year after the subjects are implanted with the subject gastric bands, and
the successful safety result is defined by an occurrence rate of hiatal hernia among the plurality of subjects, the occurrence rate ranges from about 0.5% to about 0.00001%.

17. An obesity treatment system, used in conjunction with a gastric band suitable for laparoscopic placement around a stomach of a patient to create a stoma, for determining the patient's maximum tolerance to a pressure asserted by the gastric band against the patient's stomach, the obesity treatment system comprising:
a pressure changing device configured to be coupled to the gastric band, the pressure changing device configured to repeatedly increase the pressure before the patient swallows a bolus at a step increment for every predetermined time period until a maximum tolerance event occurs; and
a pressure sensing device configured to be coupled to the gastric band, the pressure sensing device configured to detect a maximum tolerable pressure when the maximum tolerance event occurs.

18. The system of claim 17, wherein the maximum tolerance event is defined as:
the patient experiences a discomfort selected from a group consisting of lower thoracic tightness, nausea, stomach obstruction, regurgitation, reflux, pressured back throat and combinations thereof, or
the stomach of the patient induces a series of reactive pressure spikes against the gastric band and the series of reactive pressure spikes is sensed by the pressure sensing device.

19. The system of claim 17, wherein the step increment is about 0.3 psi to about 1 psi, and wherein the predetermined time period is about 1 minute to 2 minutes.

20. An obesity treatment system for collecting, processing and utilizing clinical data from a plurality of patients, each patient implanted with a gastric band suitable for laparoscopic placement around the patient's stomach to create a stoma, the system comprising:
a memory configured to store a plurality of records, each record having excess weight loss percentage and an adverse event rate relatable to at least one of the plurality of patients; and
a processor, coupled to the memory, configured to:
create a target group of records from the plurality of records based on a weight loss criterion or a safety criterion, the weight loss criterion selecting the record having the excess weight loss percentage ranges from about 20% to about 50%, the safety criterion selecting the record having the adverse event rate at about 0.05% to about 0.01%,
determine a normal distribution of the target group of records, and
calculate an optimal pressure percentage based on the normal distribution.

21. The system of claim 20, wherein each of the plurality of record comprises:
a maximum tolerable pressure value representative of the respective patient's maximum tolerance of a pressure asserted by the respective gastric band against the stomach of the respective patient;
a current gastric band pressure value indicating a current pressure asserted by the respective gastric band against the stomach of the respective patient; and
a pressure percentage value based on a ratio between the current gastric band pressure value and the maximum tolerable pressure value.

22. The system of claim 21, wherein the maximum tolerable pressure is an iso-static pressure asserted by the respective gastric band against the stomach of the respective patient.

23. The system of claim 21, wherein the maximum tolerable pressure is the pressure causing the stoma of the stomach of the respective patient to be substantially closed.

24. The system of claim 21, wherein the maximum tolerable pressure is the pressure causing the patient to experience a discomfort selected from a group consisting of lower thoracic tightness, nausea, stomach obstruction, regurgitation, reflux, pressured back throat and combinations thereof.

25. The system of claim 21, wherein the maximum tolerable pressure is the pressure causing the stomach or an esophagus of the patient to induce a series of reactive pressure spikes against the gastric band while the patient is swallowing a bolus.

26. The system of claim 21, wherein the adverse event rate is defined by a frequency of occurrence of an adverse event selected from a group consisting of hiatal hernia, bleeding, symmetrical pouch dilatation, death, serious bodily injury, and combinations thereof, the frequency of occurrence measured among a group of records having the pressure percentage values that are substantially close to one another.

27. The system of claim 20, wherein each of the plurality of records comprises:
an initial weight of the respective patient before implanted with the respective gastric band;
a current weight of the respective patient about one year after implanted with the respective gastric band; and
the excess weight loss percentage defined by a ratio of a difference between the initial weight and the current weight over a difference between the initial weight and a predefined nominal weight.

28. The system of claim 20, wherein the weight loss criterion selects the record having the excess weight loss percentage ranges from about 30% to about 40%.

29. The system of claim 20, wherein the safety criterion selects the record having the adverse event rate ranges from about 0.1% to 0.0001%.

30. A method for treating obesity, used in conjunction with a gastric band suitable for laparoscopic placement around a stomach of a patient to create a stoma, the method using real-time objective measurement and clinical data to provide optimal gastric band adjustment for the patient, the method comprising the steps of:
detecting a maximum tolerable pressure asserted by the gastric band against the stomach of the patient;
receiving an optimal pressure percentage based on a plurality of clinical data from a plurality of subjects using a plurality of subject gastric bands; and
adjusting the gastric band for asserting an optimal pressure against the stomach of the patient, the optimal pressure based on the optimal pressure percentage and the maximum tolerable pressure.

31. The method of claim 30, wherein detecting the maximum tolerable pressure comprises the steps of:
repeatedly increasing, using a pressure changing device configured to be coupled to the gastric band, a gastric band pressure, at a step increment for every predetermined time period, against the stomach of the patient before the patient swallows a bolus until a maximum tolerance event occurs; and
detecting, using a pressure sensing device configured to be coupled to the gastric band, the maximum tolerable pressure when the maximum tolerance event occurs.

32. The method of claim 31, wherein the maximum tolerance event is defined as:
the patient experiences a discomfort selected from a group consisting of stomach tightness, nausea, lower thoracic tightness, obstruction, regurgitation, reflux, pressured back throat, and combinations thereof, or
the stomach or an esophagus of the patient induces a series of reactive pressure spikes against the gastric band and the series of reactive pressure spikes is sensed by the pressure sensing device.

33. The method of claim 31, wherein the step increment is about 0.3 psi to about 1 psi, and wherein the predetermined time period is about 1 minute to 2 minutes.

34. The method of claim 30, wherein the optimal pressure percentage is determined using a method comprising the steps of:
storing, using a memory, a plurality of records, each record having an excess weight loss percentage and an adverse event rate relatable to at least one of the plurality of subjects;
creating, using a processor coupled to the memory, a target group of records from the plurality of records based on a weight loss criterion or a safety criterion, the weight loss criterion selecting the record having the excess weight loss percentage ranges from about 20% to about 50%, the safety criterion selecting the record having the adverse event rate at about 0.05% to about 0.01%;
determining, using the processor, a normal distribution of the target group of records; and
calculating, using the processor, an optimal pressure percentage based on the normal distribution.

35. The method of claim 34, wherein each of the plurality of records comprises:
an initial weight of the respective subject before implanted with the respective subject gastric band;
a current weight of the respective subject about one year after implanted with the respective subject gastric band; and
the excess weight loss percentage defined by a ratio of a difference between the initial weight and the current weight over a difference between the initial weight and a predefined nominal weight.

36. The method of claim 34, wherein the weight loss criterion selects the record having the excess weight loss percentage ranges from about 30% to about 40%.

37. The method of claim 34, wherein each of the plurality of record comprises:
a maximum tolerable pressure value representative of the respective patient's maximum tolerance of a pressure asserted by the respective gastric band against the stomach of the respective patient;
a current gastric band pressure value indicating a current pressure asserted by the respective gastric band against the stomach of the respective patient; and
pressure percentage value based on a ratio between the current gastric band pressure value and the maximum tolerable pressure value.

38. The method of claim 37, wherein the adverse event rate is defined by a frequency of occurrence of an adverse event selected from a group consisting of hiatal hernia, bleeding, symmetrical pouch dilatation, death, serious bodily injury, and combinations thereof, the frequency of occurrence measured among a group of records having the pressure percentage values that are substantially close to one another.

39. The method of claim 34, wherein the safety criterion selects the record having the adverse event rate at about 0.05%.

40. A gastric band adjustment device, used in conjunction with a gastric band suitable for laparoscopic placement around a stomach of a patient to create a stoma, the gastric band having a pressure sensing device for sensing a pressure asserted by the gastric band against the stomach of the patient and a pressure changing device for changing the pressure asserted by the gastric band, the gastric band adjustment device comprising:
a memory configured to store an optimal pressure percentage value; and
a processor, coupled to the memory, configured to:
receive the pressure detected by the pressure sensing device of the gastric band,
derive a maximum tolerable pressure based on the pressure detected by the pressure sensing device and a maximum tolerance event, the maximum tolerable pressure representative of the patient's maximum tolerance to the pressure asserted by the gastric band,
calculate an optimal pressure based on the optimal pressure percentage and the maximum tolerable pressure,
generate a pressure adjustment command based on the optimal pressure, and
remotely adjust the gastric band pressure by transmitting the pressure adjustment command to the pressure changing device of the gastric band via the external transceiver.

41. The device of claim 40, wherein the maximum tolerance event is defined as:
the patient experiences a discomfort selected from a group consisting of stomach tightness, nausea, stomach obstruction, regurgitation, and combinations thereof, or
the stomach or an esophagus of the patient induces a series of reactive pressure spikes against the gastric band and the series of reactive pressure spikes is sensed by the pressure sensing device.

42. The device of claim 40, wherein the optimal pressure percentage is based on a normal distribution of a plurality of clinical data from a plurality of subjects, each implanted with a subject gastric band, and each having:
a subject gastric band pressure based on the subject's maximum tolerable pressure;
a weight loss result ranges from about 30% to about 40% excess weight loss within about one year after the subjects are implanted with the subject gastric bands, and
the successful safety result is defined by an occurrence rate of hiatal hernia among the plurality of subjects, the occurrence rate ranges from about 0.1% to about 0.0001%.
